(19) United States
(12) Patent Application Publication
(10) Pub. No.: US 2019/0125141 A1
(43) Pub. Date: May 2, 2019

(54) SUSPENDABLE WIPE DISPENSING APPARATUS

(71) Applicants: Frank Quattromani, Lubbock, TX (US); Patricia Quattromani, Lubbock, TX (US)

(72) Inventors: Frank Quattromani, Lubbock, TX (US); Patricia Quattromani, Lubbock, TX (US)

(21) Appl. No.: 16/178,215

(22) Filed: Nov. 1, 2018

Related U.S. Application Data
(60) Provisional application No. 62/580,580, filed on Nov. 2, 2017.

(51) Int. Cl. A47K 10/38; A61K 8/02; B65D 83/08; B65D 75/46

(52) U.S. Cl. CPC A47K 10/38; A61K 8/0208; A47K 2010/389; B65D 75/46; B65D 83/0876

(57) ABSTRACT

A wipe dispensing apparatus includes a housing, a connector, a support platform, and a dispensing lid. The housing includes a top and a bottom. The connector is coupled to the housing and is for suspending the wipe dispensing apparatus from a user. The support platform is disposed within the housing and includes a support surface for supporting a plurality of wipes and a raised annular rim defining a central opening. The dispensing lid is positioned adjacent to and below the support platform. The dispensing lid includes a barrier having a dispensing aperture defined therein. The dispensing lid further includes an inner sidewall extending around and depending from the barrier towards the bottom of the housing such that the dispensing aperture is recessed from the housing bottom to facilitate shielding a portion of a wipe extending through the dispensing aperture.

### CROSS-REFERENCE TO RELATED APPLICATIONS

This application claims priority to U.S. Provisional Patent Application Ser. No. 62/580,580, filed on Nov. 2, 2017, the disclosure of which is hereby incorporated by reference in its entirety.

### BACKGROUND OF THE DISCLOSURE

The present disclosure relates generally to wipe dispensing apparatuses, and more particularly to dispensing apparatuses including a suspension mechanism for dispensing sheeted disinfecting products.

Containers for dispensing sheeted disinfecting products generally include a container configured to provide a sealed environment. Sheeted disinfecting products, particularly wipes, may be made from a variety of materials, which are dry or wet when used. Wet wipes are moistened with a variety of suitable wiping solutions. Typically, containers for holding wet wipes include a lower surface capable of collecting any solution runoff from exiting the container. Conventional containers that contain wipes are typically plastic containers, tubs, or soft-sided packages which provide a sealed environment for the wet wipes to ensure that they do not become dirty and/or dried out. To access the wipes, such containers generally include a dispensing aperture at the top of the container for dispensing the wipes from the container. These containers further generally include a moveable lid to selectively seal the dispensing aperture. Accordingly, to dispense a wipe, a user must open the lid at the top of the container and pull a wipe upwards from the dispensing aperture. By locating the dispensing aperture at the top of the container, these containers inhibit the flow of runoff solution from the wet wipes from exiting the container through the top dispensing aperture.

However, these containers are generally designed to be rested on a flat surface and are not designed for efficient dispensing while being carried by a user. Further, most wipe containers generally require that a user use two hands to dispense a wipe from the container. In particular, when dispensing a wipe from such containers, users generally must use one hand to open/close the lid and dispense the wipe, and another hand to steady or support the container while the wipe is being dispensed. As a result, in some environments, such as medical treatment facilities, wherein frequent sterilization is routinely required, employees must generally walk to a wipe dispensing container and dispense a wipe with both hands.

Accordingly, a need exists for a more portable dispensing container that is carried by the user, that allows for more convenient dispensing by the user while being carried, and that inhibits runoff of the cleaning solution from exiting the container.

This section is intended to introduce the reader to various aspects of art that may be related to various aspects of the disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### SUMMARY OF THE DISCLOSURE

In one aspect, a wipe dispensing apparatus includes a housing, a connector, a support platform, and a dispensing lid. The housing includes a top and a bottom. The connector is coupled to the housing for suspending the wipe dispensing apparatus from a user. The support platform is disposed within the housing and includes a support surface for supporting a plurality of wipes and a raised annular rim defining a central opening. The dispensing lid is positioned adjacent to and below the support platform. The dispensing lid includes a barrier having a dispensing aperture defined therein. The dispensing lid further includes an inner sidewall extending around and depending from the barrier towards the bottom of the housing such that the dispensing aperture is recessed from the housing bottom to facilitate shielding a portion of a wipe extending through the dispensing aperture.

In another aspect, a method of dispensing wipes from a wipe dispensing apparatus is provided. The wipe dispensing apparatus includes a housing having a top and a bottom, a connector coupled to the housing, and a dispensing lid defining a dispensing aperture accessible from the bottom of the housing. The housing has a plurality of wipes contained therein. The method includes suspending the wipe dispensing apparatus from a user via the connector and pulling one of the plurality of wipes downward from the dispensing lid through the dispensing aperture.

In yet another aspect, a method of assembling a wipe dispensing apparatus includes providing a housing including a top and a bottom, and a connector coupled to the housing for suspending the wipe dispensing apparatus from a user. The method further includes positioning a support platform within the housing. The support platform includes a support surface for supporting a plurality of wipes, and a raised annular rim defining a central opening. The method further includes positioning a dispensing lid adjacent to and below the support platform. The dispensing lid includes a barrier having a dispensing aperture defined therein, and an inner sidewall extending around and depending from the barrier towards the bottom of the housing such that the dispensing aperture is recessed from the housing bottom to facilitate shielding a portion of a wipe extending through the dispensing aperture.

These and other features, aspects and advantages of the present disclosure will become better understood with reference to the following description and appended claims. The accompanying drawings, which are incorporated in and constitute a part of this specification, illustrate embodiments of the disclosure and, together with the description, serve to explain the principles of the disclosure.

### BRIEF DESCRIPTION OF THE DRAWINGS

FIG. 1 is a perspective view of an exemplary wipe dispensing apparatus;

FIG. 2 is a front view of a user wearing the wipe dispensing apparatus of FIG. 1;

FIG. 3 is a side view of the user shown in FIG. 2 wearing the wipe dispensing apparatus of FIG. 1;

FIG. 4 is an exploded view of the wipe dispensing apparatus of FIG. 1;

FIG. 5 is a cross-sectional view of the wipe dispensing apparatus of FIG. 1, taken along the line 5-5 shown in FIG. 1;

FIG. 6 is a top view of a cap assembly of the wipe dispensing apparatus of FIG. 1;

FIG. 7 is a cross-sectional view of the wipe dispensing apparatus shown in FIG. 5 including a plurality of wipes positioned within the wipe dispensing apparatus;

FIG. 8 is an enlarged cross-sectional view of the wipe dispensing apparatus shown in FIG. 7; and

FIG. 9 is a perspective view of the wipe dispensing apparatus of FIG. 1, illustrating the cap assembly decoupled from the container of the wipe dispensing apparatus.

Corresponding reference characters indicate corresponding parts throughout the several views of the drawings.

### DETAILED DESCRIPTION OF THE DISCLOSURE

The present disclosure is generally related to a wipe dispensing apparatus that is configured to be conveniently attached to a user and allow for ease of use in dispensing wipes by the user while it is attached to the user. More particularly, the wipe dispensing apparatus enables a user to pull a wipe downward from the apparatus to dispense the wipe with one hand while the apparatus is suspended from the user's body. For example, in some embodiments, wipe dispensers of the present disclosure include a connector coupled generally towards the top of the apparatus from which the apparatus is suspended from the user. Accordingly, a user wears the wipe dispensing apparatus, for example, around the neck, and dispense a wipe by pulling the wipe downward from the suspended housing with a single hand.

The wipe dispensing apparatus is also configured to collect any runoff liquid (e.g., disinfecting or cleaning solution) from the wipes and inhibit leakage of the liquid from the apparatus. For example, in some embodiments, wipe dispensers of the present disclosure include a cap assembly coupled to the bottom of a container that collects liquid runoff from wet wipes stored within the container.

Further, the wipe dispensing apparatus improves accessibility of a wet wipe to the user while preventing outside contamination of the wet wipe. For example, embodiments of the wipe dispensing apparatus disclosed herein include shielding surrounding a portion of a wet wipe protruding from the container. The shielding inhibits the wet wipe from contacting foreign objects, such as clothes of a user or loose medical equipment, that might otherwise contaminate the wet wipe.

In some embodiments, the wipe dispensing apparatus is used for storing and dispensing sanitizing wipes used, for example, by medical staff or employees. For example, the wipe dispensing apparatus can be used in a wide variety of medical field areas such as general hospitals and trauma centers, military medical facilities, disaster relief medical facilities, ambulances and first responder emergency medical services, third world and non-governmental organization medical facilities, nursing and rehabilitation centers, and child care centers. Generally, the wipes stored in the containers of the present disclosure are wet wipes. Specifically, the wipes are generally soaked with an alcohol based disinfecting solution. Accordingly the wipes are configured to clean, for example, stethoscopes, bedside tables, medical equipment, office equipment, and wound care areas. However, in some embodiments, the wipe dispensing apparatus is also be used to dispense dry wipes. Other suitable wipes for use in the present disclosure include flushable moist wipes, face wipes, cosmetic wipes, household disinfecting wipes, industrial wipes, baby wipes, facial tissues, toilet tissues, napkins, and the like.

In some embodiments, the wipe dispensing apparatus is configured to dispense antimicrobial wipes that are from about 10 centimeters by 25 centimeters, from about 12 centimeters to about 20 centimeters, or from about centimeters by about 17 centimeters. In some embodiments, the wipes are from about 15.2 centimeters to about 17.1 centimeters.

In some embodiments, the wipe dispensing apparatus holds at least about 10, at least about 20, at least about 30, at least about 40, at least about 50, at least about 60, at least about 70, at least about 80, at least about 90, or at least about 100 wipes.

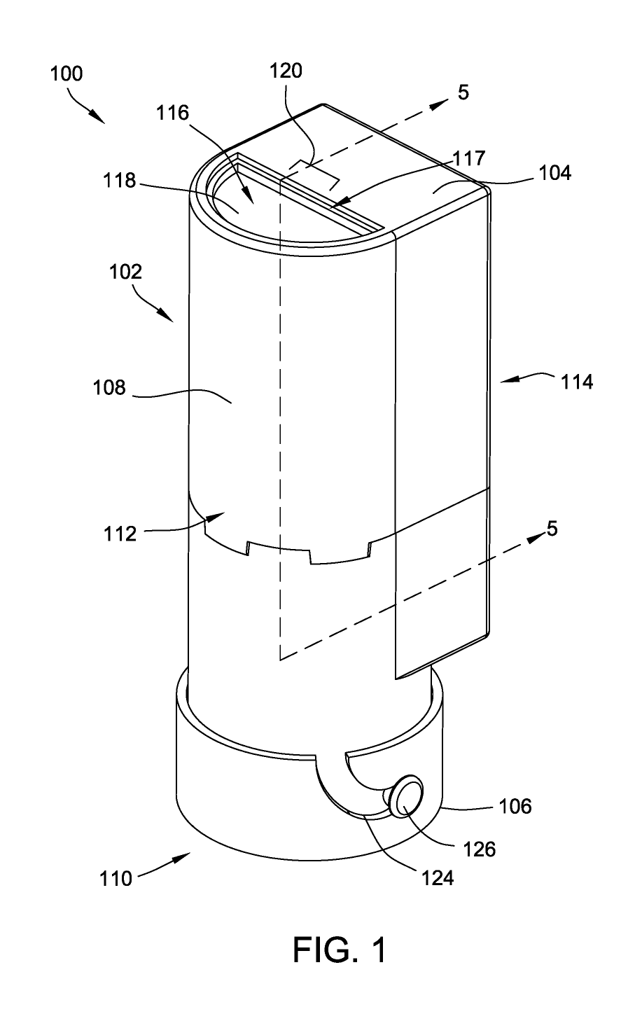

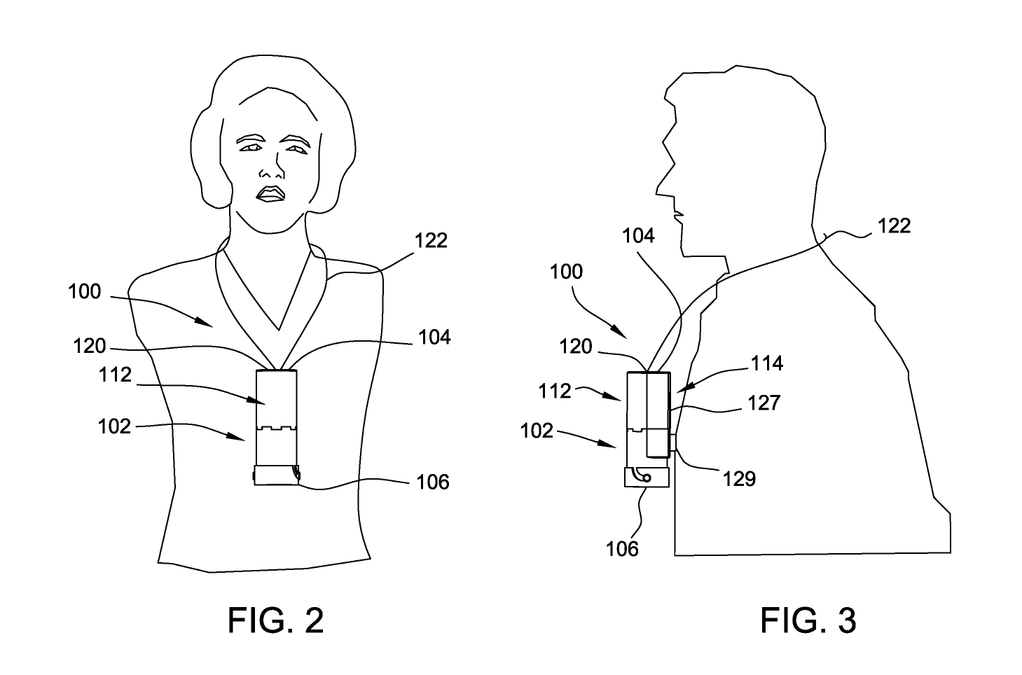

FIG. 1 is a perspective view of an exemplary wipe dispensing apparatus 100. FIG. 2 is a front view of a user wearing the wipe dispensing apparatus 100 of FIG. 1, and FIG. 3 is a side view of the user shown in FIG. 2 wearing the wipe dispensing apparatus 100 of FIG. 1.

In the illustrated embodiment, the wipe dispensing apparatus 100 generally includes a housing 102 having a top 104 and a bottom 106. The housing 102 includes a container 108 and a cap assembly 110 that defines the housing bottom 106. The cap assembly 110 is detachably coupled to the housing 102 to facilitate loading and unloading a plurality of wipes within the housing 102.

In the illustrated embodiment, the container 108 includes a front side 112 and a rear side 114 opposite the front side 112. The front side 112 includes a semi-cylindrical sidewall extending between the cap assembly 110 and the housing top 104. In the illustrated embodiment, the rear side 114 of the container 108 includes at least in part a boxed configuration protruding transversely away from the front portion 112 and rearwardly past the cap assembly 110. In other embodiments, the container 108 is entirely cylindrically shaped. In yet other embodiments, the container 108 has any suitable shape that enables it to function as described herein. For example, in some embodiments, the container 108 is ergonomically shaped to rest comfortably against a user's chest. In the illustrated embodiment, the container 108 is made of a plastic material. In particular, the container 108 is made of a fireproof plastic, such as polyvinyl chloride, polyphenylene sulfide, or a mixture of polyphenylene oxide and polystyrene. In other embodiments, the container 108 is formed of any material that enables the container 108 to function as described herein.

The container 108 is at least partially hollow and defines a cavity 116 sized and shaped to receive a plurality of wipes therein. In the illustrated embodiment, the top 104 of the housing 102 is configured to permit a wearer of the wipe dispensing apparatus 100 to monitor the quantity of remaining wipes within the housing 102 while the wipe dispensing apparatus 100 is suspended from the user. In particular, in the illustrated embodiment, the top 104 of the housing 102 defines an aperture 117 extending through the top 104 of the housing 102 and into the cavity 116. In the illustrated embodiment, the top 104 of the housing 102 includes a viewing window 118 received within the housing top 104. The viewing window 118 is configured to permit a user to view the inside of the container 108 to monitor how many wipes are remaining within the wipe dispensing apparatus 100 without having to decouple the cap assembly 110. The viewing window 118 is composed of a transparent material, such as glass or plastic. In other embodiments, the container 108 does not include a viewing window 118 and the top 104 of the housing 102 aperture is instead uncovered. In yet other embodiments, the top 104 of the housing 102 has a single uniform non-transparent surface.

The housing 102 further includes a connector 120 for suspending the wipe dispensing apparatus 100 from a user. In the illustrated embodiment, the connector 120 is located on the top 104 of the container 108. In other embodiments, the connector 120 is located at any suitable location on the housing 102 that enables the connector 120 to function as described herein. For example, in other embodiments, the connector 120 is located on a rear surface 127 (shown in FIG. 3) of the rear side 114 of the container 108. In the illustrated embodiment, the connector 120 is a loop fixedly attached to the top 104 of the housing 102 and configured to receive at least a portion of an external support attachment 122 (shown in FIG. 2) therethrough to suspend the wipe dispensing apparatus 100 from a user. In other embodiments, the connector 120 has any suitable connector configuration that enables the wipe dispensing apparatus 100 to function as described herein.

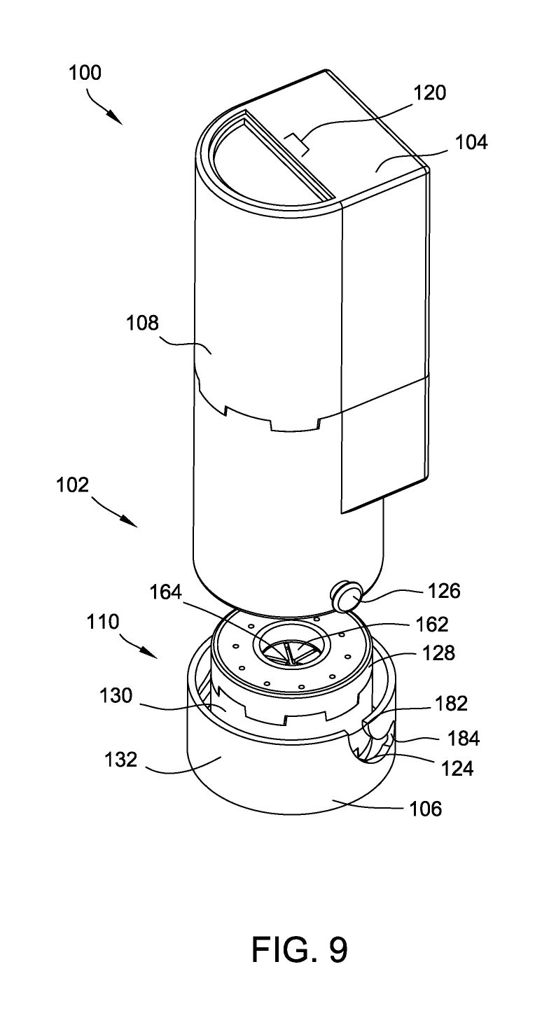

In the illustrated embodiment, the cap assembly 110 is detachably coupled to the container 108. As described in greater detail with respect to FIG. 9, detaching the cap assembly 110 from the container 108 facilitates loading and unloading a plurality of wipes into the container 108 through an opening in the bottom of the container 108. In other embodiments, the cap assembly 110 is fixedly attached to the container 108. In such embodiments, the housing 102 includes an alternative hatch (not shown) on the housing top 104 or on a side of the housing 102 for loading and unloading a plurality of wipes into the container 108.

In the illustrated embodiment, the cap assembly 110 is detachably coupled to the container 108 via a fastening mechanism that includes a pair of ramped slots 124 defined in the cap assembly 110 and pins 126 on the container 108. In particular, the pins 126 are located diametrically opposite one another on the container 108, and protrude radially outward from the container 108. The cap assembly 110 defines corresponding ramped slots 124. The ramped slots 124 are configured to receive the pins 126 therein (e.g. the ramped slots 124 are sized and shaped such that at least a portion of the pins 126 is received within the ramped slots 124). As will be described in greater detail with respect to FIG. 9, the ramped slots 124 facilitate coupling the cap assembly 110 to the container 108.

Referring to FIG. 2, the wipe dispensing apparatus 100 includes a wearable support 122 attached to the connector 120 and configured to be worn by or attached to a user. In the illustrated embodiment, the wearable support 122 is a lanyard that is looped around the neck of the user such that the wipe dispensing apparatus 100 is supported over the user's chest. In some embodiments, the wearable support 122 also includes an adjustable clip (not shown) operable to couple and decouple to the connector 120. In other embodiments, the wearable support 122 is any device that enables a user to wear the wipe dispensing apparatus 100 as described herein.

Referring to FIG. 3, the wipe dispensing apparatus 100 further includes a stabilizing connector 129 located on the rear surface 127 of the rear side 114 of the container 108. In the illustrated embodiment, the stabilizing connector 129 is positioned at a distance from the top 104 of the housing 102 of about ¾ of the height of the housing 102. The stabilizing connector 129 facilitates attaching the wipe dispensing apparatus 100 to a second location (e.g., a user's clothing) to provide additional stabilization and support of the wipe dispensing apparatus 100. In particular, the stabilizing connector 129 inhibits lateral and/or outward movement of the wipe dispensing apparatus 100 relative to the user during operation. For example, the stabilizing connector 129 inhibits movement of the wipe dispensing apparatus 100 when a user pulls a wipe to dispense the wipe from the housing bottom 106. Thus, the stabilizing connector 129 aids a user in dispensing a wipe using only a single hand by eliminating the need to stabilize the housing 102 with another hand while dispensing the wipe.

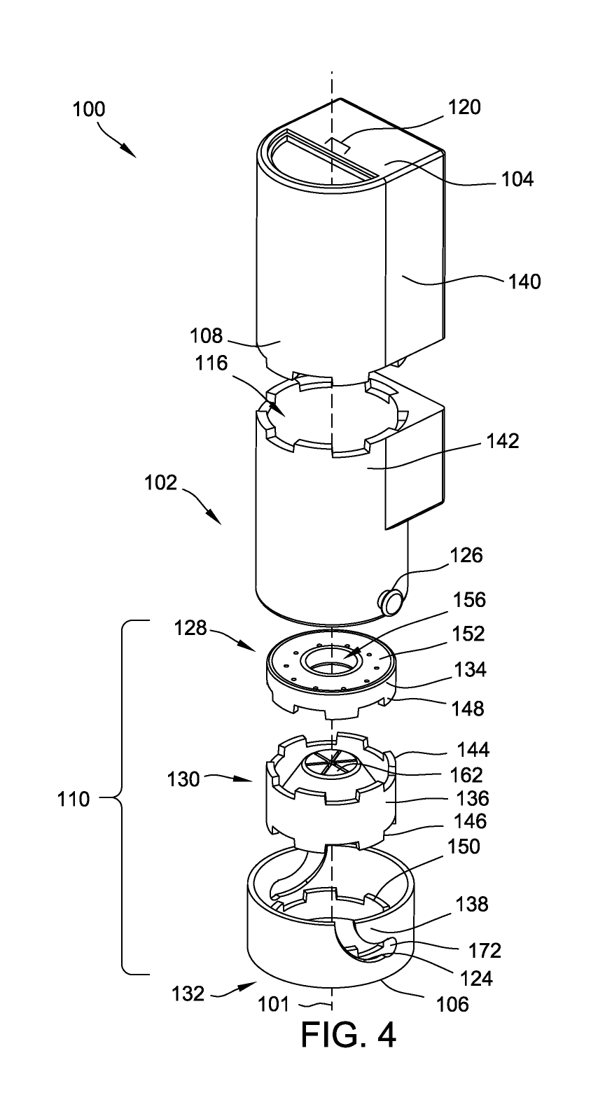

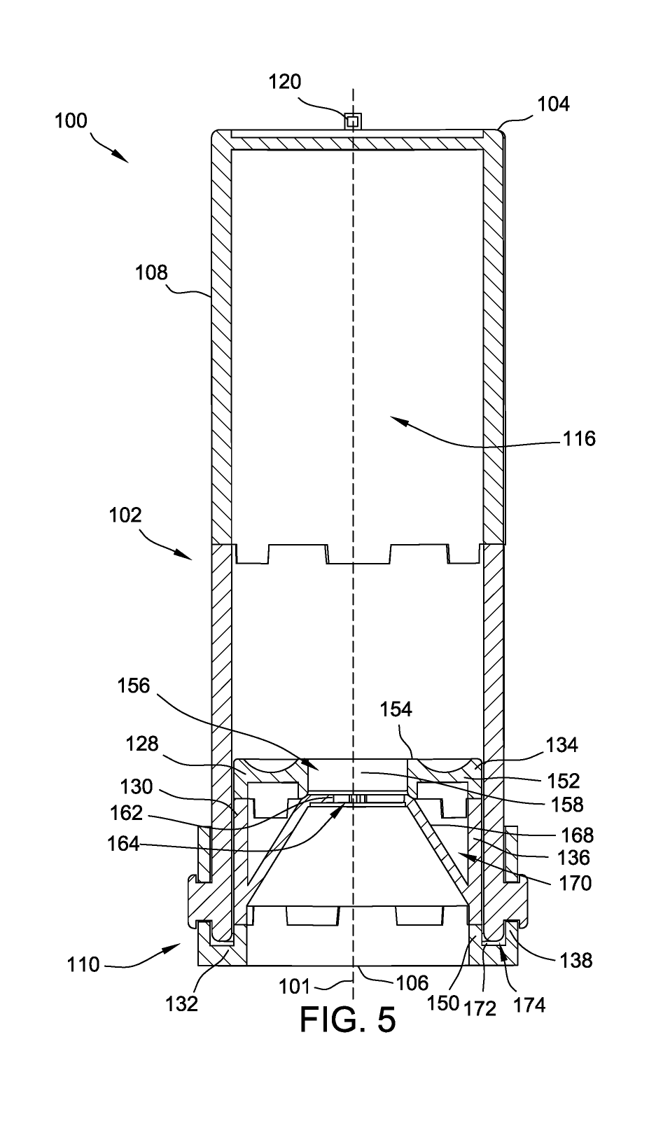

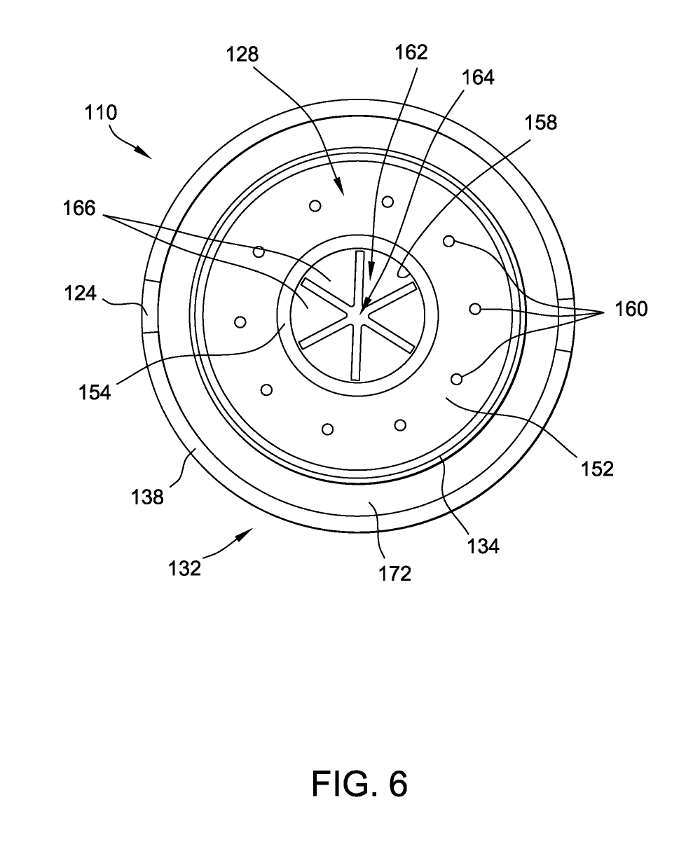

FIG. 4 is an exploded view of the wipe dispensing apparatus 100 of FIG. 1. FIG. 5 is a cross-sectional view of the wipe dispensing apparatus 100 of FIG. 1, taken along the line 5-5 shown in FIG. 1. Referring to FIG. 4, a longitudinal axis 101 of the housing 102 is shown extending from the top 104 of the housing 102 through the bottom 106 of the housing 102.

In the illustrated embodiment, the cap assembly 110 includes a support platform 128, a dispensing lid 130, and an outer cover 132. In the illustrated embodiment, the support platform 128, dispensing lid 130, and outer cover 132 are shown in a stacked configuration and positioned in longitudinal alignment with one another. In the illustrated embodiment, the support platform 128, dispensing lid 130, and outer cover 132 are each shaped to have a circular perimeter. In particular, in the illustrated embodiment, the support platform 128 includes an outer sidewall 134, the dispensing lid 130 includes an outer sidewall 136, and the outer cover includes an outer sidewall 138. The outer sidewalls 134, 136, 138 are each radially spaced from the longitudinal axis 101 and respectively define the circular perimeters of the support platform 128, the dispensing lid 130, and the outer cover 132. In the illustrated embodiment, the outer sidewall 138 of the outer cover 132 also defines an outer perimeter of the cap assembly 110. In other words, the outer sidewall 138 of the outer cover 132 is radially spaced a greater distance from the longitudinal axis 101 than the support platform 128 and dispensing lid outer sidewalls 134, 136. In other embodiments, the support platform 128, dispensing lid 130, and outer cover 132 are shaped in any manner that enables the cap assembly 110 to function as described herein. For example, in other embodiments, the support platform 128, dispensing lid 130, and the outer cover 132 are shaped to have outer perimeters corresponding to the shape of the container 108.

In the illustrated embodiment, the support platform 128 is attached to the dispensing lid 130 by adhesive, and the dispensing lid 130 is attached to the outer cover 132 by adhesive. The outer cover 132 is detachably coupled to the housing 102 at the housing bottom 106 by engagement between the slots 124 of the outer cover 132 and the pins 126 of the housing 102. The outer cover 132 secures the dispensing lid 130 and the support platform 128 to the housing 102 such that the dispensing lid 130 and the support platform 128 are each removed from the housing 102 upon detaching the outer cover 132 from the housing 102.

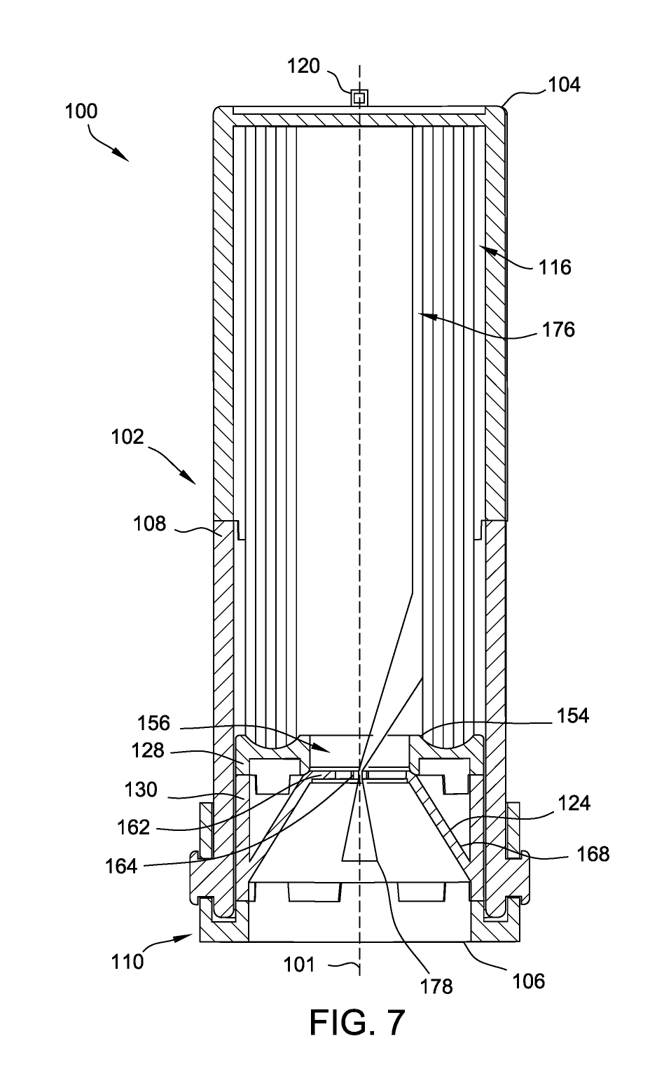

In the illustrated embodiment, the container 108 includes an upper portion 140 and a lower portion 142 formed from separate pieces and connected to one another via a castellated connection. In other embodiments, the container 108 is a single unitary piece. The container 108 is hollowed to define the cavity 116 and includes an open bottom (not shown) of the lower portion 142 to facilitate loading and unloading a plurality of wipes 176 (shown in FIG. 7) into the cavity 116. In the illustrated embodiment, the container 108 is hollowed such that the cavity 116 is cylindrical. In other embodiments, the cavity 116 has any suitable shape that enables the wipe dispensing apparatus 100 to function as described herein.

In the illustrated embodiment, the dispensing lid 130 attaches to the support platform 128 and the outer cover 132 via a top and bottom castellated connection rims 144, 146. The castellated connection rims 144, 146 are located on the outer sidewall 136 of the dispensing lid 130. The castellated connection rims 144, 146 of the dispensing lid 130 include castellations circumferentially spaced around the outer sidewall 136 that define recesses spaced between the respective castellations. The support platform 128 further includes a castellated connection 148 on the outer sidewall 134. Accordingly, the rectangular projections and recesses of the top castellated connection 144 of the dispensing lid 130 are sized and spaced to correspond to respective rectangular projections and recesses of the castellated connection 148 on the support platform 128 such that the dispensing lid 130 is adhesively attached to the support platform 128 by aligning each rectangular projection of the top castellated connection 144 of the dispensing lid 130 with a corresponding recess of the castellated connection 148 of the support platform 128. The outer cover 132 further includes a castellated connection 150 spaced radially inward from the outer sidewall 138. Likewise, the dispensing lid 130 is adhesively attached to the outer cover 132 by aligning each rectangular projection of the bottom castellated connection 146 of the dispensing lid 130 with each recess of the castellated connection 150 of the outer cover 132. In other embodiments, the castellated rims are shaped in any manner that enables the cap assembly 110 to function as described herein. In yet further embodiments, the outer cover 132, the dispensing lid 130, and the support platform 128 are attached to one another in any manner that enables the wipe dispensing apparatus 100 to function as described herein. For example, in other embodiments, the outer cover 132, dispensing lid 130, and the support platform 128 are unitarily formed as a single piece.

In the illustrated embodiment, the outer sidewalls 134, 136 of the support platform 128 and the dispensing lid 130 are each radially spaced an equal distance from the longitudinal axis 101 and are at least small enough to fit within the cavity 116 of the container 108. In the illustrated embodiment, the outer cover 132 is sized such that the diameter of the outer cover 132 is greater than the outer diameter of the container 108 to facilitate positioning at least a portion of the container 108 within the outer cover 132.

The support platform 128 is annular and includes a support surface 152 facing the housing top 104 for supporting a plurality of wipes 176. Referring to FIG. 5, the support surface 152 also includes a raised annular rim 154 located radially inward of the outer sidewall 134 and defining a central opening 156. A central opening sidewall 158 is oriented generally transversely to the support surface 152 and extends longitudinally to define the central opening 156 through the support platform 128. The support surface 152 extends circumferentially between the raised annular rim 154 and the outer sidewall 134.

FIG. 6 is a top view of the cap assembly 110 of the wipe dispensing apparatus 100 of FIG. 1. In the illustrated embodiment, a plurality of apertures 160 are defined within the support surface 152 approximately radially midway between the outer sidewall 134 and the raised annular rim 154. The plurality of apertures 160 are circumferentially spaced equidistantly from one another around the support surface 152. In other embodiments, the plurality of apertures 160 are spaced in any manner that enables the support platform 128 to function as described herein. For example, in other embodiments, only a single aperture (not shown) is defined within the support surface 152. In such embodiments, the single aperture is a slit extending circumferentially around the support platform 128. In the illustrated embodiment, each aperture 160 of the plurality of apertures 160 extends through the support platform 128 to provide fluid communication between the support surface 152 and the dispensing lid 130. Referring to FIG. 5, the support surface 152 has a concave contour sloping downward both radially outward from the raised annular rim 154 and radially inward from the outer sidewall 134. The bottom or lowest point of the support surface 152 is located approximately midway between the raised annular rim 154 and the outer sidewall 134. In particular, in the illustrated embodiment, the plurality of apertures 160 are each defined at the bottom of the support surface 152. Accordingly, the concave contour of the support surface 152 acts as a gutter directing fluid towards the plurality of apertures 160 to facilitate drainage of fluid from the support platform 128 through the plurality of apertures 160. In other embodiments, the support surface 152 defines multiple radially aligned apertures 160 spanning the concave contour of the support surface 152.

In the illustrated embodiment, the dispensing lid 130 is positioned adjacent to and below the support platform 128. The dispensing lid 130 includes a barrier 162 oriented transversely to the central opening sidewall 158. The barrier 162 defines a dispensing aperture 164 to facilitate the dispensing of a wipe through the barrier 162. In particular, in the illustrated embodiment, the barrier 162 includes a plurality of individual barrier pieces 166 configured to separate two connected wipes (e.g., a leading wipe and a trailing wipe) from one another as they are dispensed and to inhibit additional wipes from coming out of the housing 102. In the illustrated embodiment, the barrier pieces 166 include a plurality of triangular pieces extending radially inward from an outer edge of the barrier 162. Each of the barrier pieces 166 is separated from adjacent barrier pieces 166 by a slit. Each barrier piece 166 is constructed of a resiliently flexible material such that the barrier pieces 166 are capable of flexing or deflecting downward in response to frictional engagement between a wipe and the barrier pieces 166 resulting from the movement of a wipe through the dispensing aperture 164.

Referring to FIG. 5, the barrier 162 is located in longitudinal alignment with the central opening 156 of the support platform 128 and has a diameter approximately equal to the diameter of the central opening 156. More specifically, in the illustrated embodiment, the dispensing aperture 164 of the barrier 162 is located at the center of the central opening 156. Further, the barrier 162 contacts and is surrounded by the central opening sidewall 158 of the support platform 128 to prevent wipes from becoming trapped between the dispensing lid 130 and the support platform 128.

In some embodiments, the dispensing aperture 164 of the barrier 162 is positioned relative to the connector 120 such that a wipe is dispensed from the dispensing aperture 164 in a direction substantially parallel to the direction in which the housing 102 is supported by the connector 120. That is, the dispensing aperture 164 is positioned relative to the connector 120 to facilitate the connector 120 applying a force on the housing 102 substantially opposite the force applied on the housing 102 from dispensing a wipe through the dispensing aperture 164. The configuration of the dispensing aperture 164 relative to the connector 120 facilitates stabilizing the housing 102 on the user when a wipe is dispensed from the dispensing aperture. More specifically, locating the dispensing aperture 164 to allow for dispensing of a wipe in a direction parallel to the direction in which the housing 102 is supported by the connector 120 allows for the dispensing of a wipe from the housing 102 without imparting lateral force on the housing 102. Accordingly, in some embodiments, a user dispenses a wipe from the wipe dispensing apparatus 100 with a single hand, without needing to use a second hand to stabilize the housing 102. In the illustrated embodiment, the dispensing aperture is longitudinally aligned with the connector 120. In other embodiments, the connector 120 and the dispensing aperture 164 are located relative to one another in any suitable configuration that enables the wipe dispensing apparatus 100 to function as described herein.

The dispensing lid 130 also includes an inner sidewall 168 extending around and depending from the barrier 162 towards the bottom 106 of the housing 102 such that the dispensing aperture 164 is recessed from the housing bottom 106. As will be described in greater detail below, the dispensing aperture 164 being recessed from the housing bottom 106 facilitates shielding a portion of a wipe extending through the dispensing aperture 164. In the illustrated embodiment, the inner and outer sidewalls 168, 136 of the dispensing lid 130 cooperatively define a first fluid reservoir, indicated generally at 170.

In the illustrated embodiment, the inner sidewall 168 of the dispensing lid 130 is a single continuous sidewall obliquely oriented to the outer sidewall 136 and the barrier 162. The inner sidewall 168 extends around the barrier 162 within the outer sidewall 136. The inner sidewall 168 tapers conically upwards from the outer sidewall 136 to the barrier 162 such that the inner sidewall 168 and the barrier 162 cooperatively have an open-bottomed frustoconical shape. In other embodiments, the inner sidewall 168 is shaped in any manner that enables the dispensing lid 130 to function as described herein.

Referring to FIG. 4, the outer cover 132 is annular with the outer sidewall 138 and the interior castellated connection 150 extending longitudinally. The interior castellated connection 150 is configured to engage the lower castellated connection 146 of the dispensing lid 130 as described above. Furthermore, as shown in FIG. 5, an upper surface 172 of the outer cover 132 extends radially between the outer sidewall 138 and the interior castellated connection 150. The upper surface 172 is transversely oriented relative to the outer sidewall 138 and the interior castellated connection 150. The upper surface 172, the interior castellated connection 150, and the outer sidewall 138 of the outer cover 132 cooperatively define a second reservoir 174. The outer sidewalls 134, 136 of the support platform 128 and the dispensing lid 130 are spaced radially inward from the container 108 such that the second reservoir 174 is in fluid communication with the cavity 116 of the container 108, and liquid from the cavity (e.g., liquid runoff from the wipes) flows between outer sidewalls 134, 136 and the container 108 into the second reservoir 174.

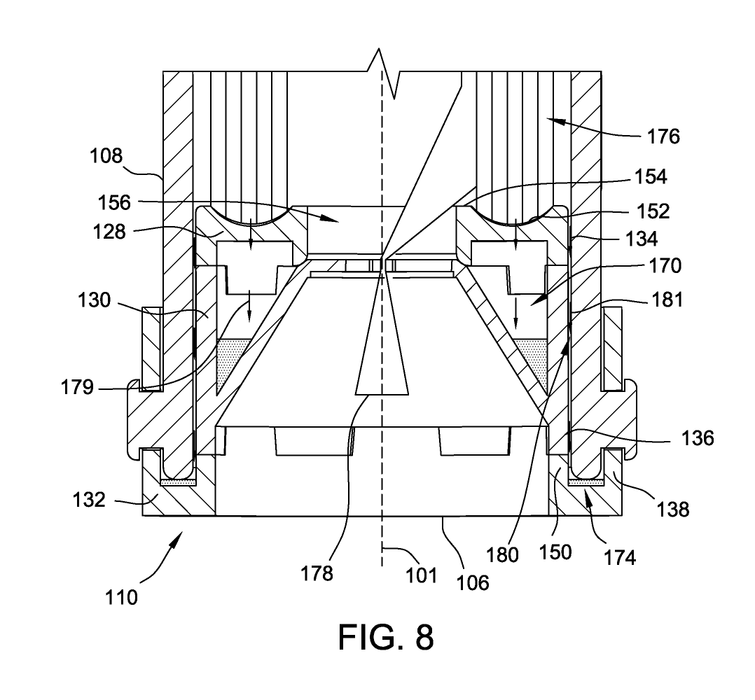

FIG. 7 is a cross-sectional view of the wipe dispensing apparatus 100 shown in FIG. 5 including a plurality of wipes 176 loaded in the wipe dispensing apparatus 100. FIG. 8 is an enlarged cross-sectional view of the wipe dispensing apparatus 100 shown in FIG. 7 including fluid and arrows 179, 181 indicating fluid flow. In the illustrated embodiment, the wipes 176 are shown loaded into the cavity 116 of the container 108. Each of the wipes includes a leading end 178 and a trailing end (not labeled). In the illustrated embodiment, the wipes 176 are arranged as a single roll of wipes extending circumferentially around the central opening 156. In particular, the single roll of wipes includes a series of equidistant spaced predetermined perforated landmarks (not shown), separating the trailing end of a wipe from the leading end of the trailing wipe. The predetermined perforated landmarks are arranged to facilitate separating each wipe from the plurality of wipes 176. In the illustrated embodiment, the wipes 176 are arranged to unravel about the central opening 156 with later order wipes wrapped radially outward from the central opening 156. Accordingly, the portion of the wipe extending through the central opening 156 and the dispensing aperture 164 defined in the barrier 162 is the leading end 178 of the most radially inward wrapped wipe. In other embodiments, the plurality of wipes 176 are precut from one another and positioned within the container 108 to overlap each other. For example, in other embodiments each trailing end of the wipes overlaps the leading end of a trailing wipe.

In the illustrated embodiment, the leading end 178 of the most radially inward wrapped wipe is supported below the dispensing aperture 164 and above the housing bottom 106 such that the wipe is shielded by the inner sidewall 168 of the dispensing lid 130. In use, when the wipe dispensing apparatus 100 is suspended from a user via the connector 120, to dispense the wipe from the wipe dispensing apparatus 100, a user reaches upward with one hand into the recessed region of the wipe dispensing apparatus 100 and grasp the leading end 178 of a wipe protruding through the barrier 162. The user then pulls the wipe downward from the dispensing lid 130 and through the dispensing aperture 164 in the barrier 162. As the wipe is pulled through the barrier 162, the wipe unravels from the roll of wipes 176 about the annular rim 154 of the support platform 128. As the perforated edge of the wipe passes through the barrier 162, the barrier pieces 166 (shown in FIG. 6) engage the perforated edge thereby causing the wipe to tear from the trailing wipe and dispense from the housing 102. Accordingly, as the wipe is removed from the housing bottom 106, the leading end 178 of the trailing wipe is supported above the bottom 106 of the housing 102 and is shielded by the inner sidewall 168 of the dispensing lid 130.

Referring to FIG. 8, the plurality of wipes 176 shown in the illustrated embodiment are wet wipes soaked in a cleaning solution. In use, in some embodiments, some of the cleaning solution runs off the plurality of wipes 176 and collect on the support surface 152 of the support platform 128. In the illustrated embodiment, runoff cleaning solution is shown collected in the first reservoir 170 defined between the dispensing lid 130 and the support platform 128. A first set of fluid flow arrows 179 shows the path of the runoff cleaning solution from the support surface 152 through the plurality of apertures 160 (shown in FIG. 6) and into the first reservoir 170.

In the illustrated embodiment, a second set of fluid arrows 181 shows an additional flow of cleaning solution from the support surface 152 into the second reservoir 174 defined within the outer cover 132. In particular, when cleaning solution accumulates on the support surface 152, in some embodiments, it overflows from the support surface 152 into a longitudinally extending gap 180 defined between the container 108 and the outer sidewalls 134, 136 of the support platform 128 and the dispensing lid 130. The cleaning solution is shown collected in the second reservoir 174 defined between the outer sidewall 138 and castellated connection 150 of the outer cover 132. Accordingly, the gap 180 and the second reservoir 174 provide an additional path for overflow cleaning solution to lessen any overflow that might otherwise flow over the raised annular rim 154 and leak out of the housing 102 through the dispensing aperture 164. The second reservoir 174 and gap 180 provide a backup for overflow cleaning solution, for example, if the plurality of apertures 160 (shown in FIG. 6) in the support surface 152 become blocked by the plurality of wipes 176 resting on the support surface 152.

FIG. 9 is a perspective view of the wipe dispensing apparatus 100 of FIG. 1 showing the cap assembly 110 decoupled from the container 108. In the illustrated embodiment, the ramped slots 124 of the outer cover 132 each include a free end 182 and a locking portion 184 opposite the free end 182. In the illustrated embodiment, the ramped slots 124 are sloped generally downward and extend circumferentially (counter-clockwise in the illustrated embodiment) from the free ends 182 of the ramped slots 124 to the locking portions 184. In other embodiments, the ramped slots 124 extend in any orientation or direction that enables the ramped slots 124 to function as described herein. In the illustrated embodiment, the ramped slots 124 are each sized such that the respective pins 126 each engage the ramped slots 124 at the locking portion 184 to resist decoupling the cap assembly 110 from the container 108. In other words, the ramped slots 124 are configured to inhibit unlocking rotational movement of the pins 126 when the pins 126 are received within the locking portion 184 of the ramped slots 124. In the illustrated embodiment, counterclockwise rotation of the cap assembly 110 relative to the container 108 is unlocking rotational movement. In other embodiments, clockwise rotation of the cap assembly 110 relative to the container 108 is unlocking rotational movement. In further embodiments, the cap assembly 110 is unlocked from the container 108 via any movement that enables the cap assembly 110 to function as described herein.

In use, to fill the housing 102 with a plurality of wipes, a user detaches the cap assembly 110 from the container 108 to expose an open end of the container 108. In the illustrated embodiment, a user rotates the outer cover 132 relative to the container 108 to provide unlocking rotational motion of the outer cover 132. In particular, in the illustrated embodiment, the user rotates the outer cover 132 forty-five degrees relative to the container 108. The unlocking rotational motion causes the pins 126 to snap out from the locking portion 184. Due to the incline of the ramped slots 124, further unlocking rotational movement of the outer cover 132 relative to the container 108 causes the outer cover 132 to move longitudinally downward from the container 108 until the pins 126 reach the free end 182 of the ramped slots 124. In some embodiments, once the free end 182 of the ramped slots 124 is moved past the pins 126, the cap assembly 110 is fully separated from the container 108. The user then fills the container 108 with a plurality of wipes by placing the plurality of wipes 176 into the container 108 through the open end of the container 108.

In some embodiments, the user then feeds the leading end 178 (shown in FIG. 8) of the most radially inward wipe through the open end of the container 108 and the dispensing aperture 164 defined in the barrier 162 so that the leading end 178 of the wipe will be accessible for the user to pull after the cap assembly 110 is reattached to the container 108. In other embodiments, the leading end 178 of the first wipe in the roll includes a guiding device, such as for example, a string, that is smaller than the wipes and configured to be pre-fed through the dispensing aperture 164 in the barrier 162 such that the user attaches the cap assembly 110 to the container 108 before the wipe is fed through the dispensing aperture 164. To reattach the cap assembly 110 to the container 108, the user guides the free ends 182 of the ramped slots 124 to be in alignment with the pins 126 of the container 108. The user then twists the cap assembly 110 by providing locking rotational motion to guide the pins 126 of the container 108 to the locking portion 184 of the ramped slots 124.

Embodiments of wipe dispensing apparatus described herein are conveniently attached to a user and allow for improved ease of use in dispensing wipes as compared to previous wipe dispensers. For example, embodiments described herein enable a user to pull a wipe downward from the apparatus to dispense the wipe with one hand while the apparatus is attached to or suspended from the user's body. Moreover, the wipe dispensing apparatus collects runoff cleaning solution from the wipes to inhibit leakage of the cleaning solution from the apparatus. For example, wipe dispensers of the present disclosure include one or more fluid reservoirs that collect liquid runoff from wet wipes stored within the container and inhibit liquid from escaping from a downward facing dispensing aperture. Additionally, embodiments of the wipe dispensing apparatus described herein improve accessibility of a wet wipe to the user while preventing outside contamination of the wet wipe by including shielding surrounding a portion of a wet wipe protruding from the container.

When introducing elements of the present disclosure or suitable embodiment(s) thereof, the articles “a”, “an”, “the”, and “said” are intended to mean that there are one or more of the elements. The terms “comprising,” “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A wipe dispensing apparatus comprising:
a housing comprising a top and a bottom;
a connector coupled to said housing for suspending said wipe dispensing apparatus from a user;
a support platform disposed within said housing and comprising:
a support surface for supporting a plurality of wipes; and
a raised annular rim defining a central opening; and
a dispensing lid positioned adjacent to and below said support platform, said dispensing lid comprising:
a barrier having a dispensing aperture defined therein; and
an inner sidewall extending around and depending from said barrier towards said bottom of said housing such that said dispensing aperture is recessed from said housing bottom to facilitate shielding a portion of a wipe extending through said dispensing aperture.

2. The wipe dispensing apparatus of claim 1, wherein said housing defines a longitudinal axis, and wherein said dispensing aperture is longitudinally aligned with said central opening.

3. The wipe dispensing apparatus of claim 1, wherein said housing defines a cavity for receiving and storing the plurality of wipes.

4. The wipe dispensing apparatus of claim 1, wherein said dispensing lid comprises an outer sidewall disposed radially outward from said inner sidewall, wherein said inner and outer sidewalls cooperatively define a fluid reservoir.

5. The wipe dispensing apparatus of claim 4, wherein said dispensing lid is coupled to said support platform at said outer sidewall, and wherein said inner sidewall tapers conically upwards from said outer sidewall to said barrier.

6. The wipe dispensing apparatus of claim 4, wherein said support surface has a plurality of apertures defined therein to permit fluid flow through said support platform and into said fluid reservoir.

7. The wipe dispensing apparatus of claim 1, wherein said housing defines a longitudinal axis, and wherein said dispensing aperture is longitudinally aligned with said connector.

8. The wipe dispensing apparatus of claim 1 further comprising an outer cover comprising a ramped slot, said outer cover detachably coupled to said housing at a pin protruding outwardly from said housing and configured to engage said ramped slot of said outer cover.

9. The wipe dispensing apparatus of claim 8, wherein said outer cover secures said dispensing lid and said support platform to said housing such that said dispensing lid and said support platform are removable from said housing upon detaching said outer cover from said housing.

10. The wipe dispensing apparatus of claim 8, wherein said outer cover extends around and below said dispensing lid.

11. A method of dispensing wipes from a wipe dispensing apparatus including a housing having a top and a bottom, a connector coupled to the housing, and a dispensing lid defining a dispensing aperture accessible from the bottom of the housing, the housing having a plurality of wipes contained therein, the method comprising:
suspending the wipe dispensing apparatus from a user via the connector; and
pulling one of the plurality of wipes downward from the dispensing lid through the dispensing aperture.

12. The method of claim 11, wherein the wipe dispensing apparatus further includes a support platform disposed within the housing, the method further comprising:
detaching the support platform from the bottom of the housing;
loading a plurality wipes into the housing through the bottom of the housing; and
securing the support platform to the housing after loading the plurality of wipes such that the plurality of wipes are supported by the support platform when the wipe dispensing apparatus is suspended from the user.

13. The method of claim 12, wherein the wipe dispensing apparatus further includes an outer cover detachably coupled to the housing bottom and fixedly attached to the support platform, wherein detaching the support platform from the bottom of the housing includes rotating the outer cover relative to the housing.

14. The method of claim 12 further comprising:
feeding the one of the plurality of wipes through the dispensing lid such that the wipe is accessible for the user to pull after the support platform is secured to the housing.

15. A method of assembling a wipe dispensing apparatus comprising:
providing a housing including a top and a bottom, and a connector coupled to the housing for suspending the wipe dispensing apparatus from a user;
positioning a support platform within the housing, the support platform including a support surface for supporting a plurality of wipes, and a raised annular rim defining a central opening; and
positioning a dispensing lid adjacent to and below the support platform, the dispensing lid including a barrier having a dispensing aperture defined therein, and an inner sidewall extending around and depending from the barrier towards the bottom of the housing such that the dispensing aperture is recessed from the housing bottom to facilitate shielding a portion of a wipe extending through the dispensing aperture.

16. The method of claim 15, wherein the dispensing lid comprises an outer sidewall disposed radially outward from the inner sidewall, wherein the inner and outer sidewalls cooperatively define a fluid reservoir.

17. The method of claim 16, wherein the dispensing lid is coupled to the support platform at the outer sidewall, and wherein the inner sidewall tapers conically upwards from the outer sidewall to the barrier.

18. The method of claim 16, wherein the support surface has a plurality of apertures defined therein to permit fluid flow through the support platform and into the reservoir.

19. The method of claim 15 further comprising:
detachably coupling an outer cover to the housing at the bottom such that the outer cover secures the dispensing lid and the support platform to the housing such that the dispensing lid and the support platform are removable from the housing upon detaching the outer cover from the housing.

20. The method of claim 19, wherein the outer cover is fixedly coupled to the dispensing lid and the support platform.
